(19) United States
(12) Patent Application Publication
(10) Pub. No.: US 2024/0002953 A1
(43) Pub. Date: Jan. 4, 2024

(54) METHOD FOR DETECTING POLYNUCLEOTIDE VARIATIONS

(71) Applicant: AnchorDx Medical Co., Ltd., Guangzhou (CN)

(72) Inventors: Zhiwei CHEN, Guangzhou (CN); Jianbing FAN, Guangzhou (CN)

(21) Appl. No.: 18/335,453

(22) Filed: Jun. 15, 2023

Related U.S. Application Data
(63) Continuation of application No. PCT/CN2021/086104, filed on Apr. 9, 2021.

(30) Foreign Application Priority Data
Dec. 15, 2020 (CN) 202011479880.7
Mar. 12, 2021 (CN) 202110269166.3

(51) Int. Cl. C12Q 1/6886; G16B 20/20; G06N 20/00

(52) U.S. Cl. CPC C12Q 1/6886; G16B 20/20; G06N 20/00; C12Q 2600/156; C12Q 2600/154

(57) ABSTRACT

The present invention relates to a method for detecting polynucleotide variations by putative methylation and hydroxymethylation surrogate markers. The method comprises the following steps of: 1) isolating a polynucleotide from a biological sample; 2) identifying and characterizing methylation and/or hydroxymethylation biomarkers; and 3) identifying relevant methylation and/or hydroxymethylation markers or building a model according to candidate markers to infer and/or determine the polynucleotide variations. As a non-invasive adjuvant diagnostic method for precision cancer medicine, the method for detecting polynucleotide variations of the present invention is particularly effective for the identification of surrogate biomarkers in blood. The detection of the polynucleotide variations in the present invention can be used in the detection, prediction, precise treatment or postoperative monitoring of diseases.

### TECHNICAL FIELD

The present invention belongs to the field of biotechnology and specifically relates to a method for detecting polynucleotide variations by methylation and hydroxymethylation surrogate markers.

### BACKGROUND OF THE INVENTION

Polynucleotide variations, i.e. somatic mutations (including detection and quantification of single nucleotide variations (SNVs), insertions and deletions (InDels), fusions, and copy number variations (CNVs)) are of importance in molecular biology and medical applications, such as diagnostics and prognostics. “Personalized medicine” is increasingly known as “precision medicine”, and its core objective is to combine patient's specific gene information with a therapeutic regimen matched with the patient's gene characteristics [Ashley, E. A. J. N. R. G., Towards precision medicine. 2016. 17(9): p. 507.]. To achieve such an objective, a reliable genetic testing must be established to reliably determine the genetic status of relevant genes, e.g. diseases caused by genetic alterations (e.g., polynucleotide variations) or epigenetic changes (e.g., DNA methylation and DNA hydroxymethylation).

Early detection and monitoring of genetic disorders (e.g., cancer) in the level of genetic aberrations (e.g., SNVs, InDels, fusions and CNVs) are usually necessary for the appropriate treatment, genetic counseling and prophylaxis strategies of patients [Garofalo, A., et al., The impact of tumor profiling approaches and genomic data strategies for cancer precision medicine. 2016. 8(1): p. 1-10.]. Several methods for direct detection of genetic variations have been developed at present, such as polymerase chain reaction (PCR), multiplex ligation probe amplification (MLPA) and DNA chip technology [Jameson, J. L., D. L. J. O. Longo, and g. survey, Precision medicine—personalized, problematic, and promising. 2015. 70(10): p. 612-614.]. In recent years, next generation sequencing (NGS) and other techniques have been emerged and greatly improved, and are capable of achieving rapid, high-throughput and high-accuracy detection of multiple genetic variations [Dong, L., et al., Clinical next generation sequencing for precision medicine in cancer. 2015. 16(4): p. 253-263.].

The reliability of a detection method depends greatly on the types of biological samples, such as blood and tissues. Liquid biopsy is a sample detection method for monitoring free nucleic acids derived from different types of body fluids. Compared with methods utilizing tissue specimens, the method has the advantages of low invasiveness, real-time monitoring during treatment, easy and frequent detection, and decrease and/or elimination of disease heterogeneity [Rossi, G. and M. J. C. r. Ignatiadis, Promises and pitfalls of using liquid biopsy for precision medicine. 2019. 79(11): p. 2798-2804.]. However, due to the limited amount of nucleic acids in body fluids, there are always problems such as limited sensitivity and low signal-to-noise ratio in conventional detection methods [Wang, J., et al., Application of liquid biopsy in precision medicine: opportunities and challenges. 2017. 11(4): p. 522-527.]. Therefore, there is a need in the art for an improved technology and/or system for detecting genetic variations, which uses an alternative strategy, for example, a surrogate biomarker for detection and monitoring of a disease.

Methylation or hydroxymethylation of CpG sites is an epigenetic regulatory factor of gene expression, and often results in gene silencing or activation. Extensive disturbance of DNA methylation has been noted in a variety of diseases, particularly in cancer, and it will lead to alterations in gene regulation, thus promoting the development of cancer [Das, P. M. and R. J. J. o. c. o. Singal, DNA methylation and cancer. 2004. 22(22): p. 4632-4642.]. Certain changes in methylation have been repeatedly found in nearly all specific types of cancers. It has been demonstrated that these changes have great potential as biomarkers for early screening, therapeutic response prediction and prognosis. Thus, it is reasonable and feasible to use a methylation or hydroxymethylation biomarker as a surrogate to detect polynucleotide variations, thereby avoiding the limitations in conventional detection technologies.

### SUMMARY OF THE INVENTION

A technical solution for achieving the above objective is as follows.

A method of detecting polynucleotide variations comprises the following steps of:
1) isolating a polynucleotide from a biological sample;
2) identifying and characterizing methylation and/or hydroxymethylation biomarkers; and
3) identifying relevant methylation and/or hydroxymethylation markers or building a model according to candidate markers to infer and/or determine the polynucleotide variations.

In some embodiments, the polynucleotide comprises DNA.

In some embodiments, the polynucleotide comprises RNA.

In some embodiments, the polynucleotide variations comprise single nucleotide variations (SNVs).

In some embodiments, the polynucleotide variations comprise insertions and/or deletions (InDels).

In some embodiments, the polynucleotide variations comprise fusions.

In some embodiments, the polynucleotide variations comprise copy number variations (CNVs).

In some embodiments, the biological sample comprises a biological fluid sample, such as blood, serum, plasma, vitreous, sputum, urine, tears, sweat, and saliva.

In some embodiments, the biological sample comprises a tissue sample.

In some embodiments, the biological sample comprises a cell line sample.

In some embodiments, the isolating comprises phenol and/or chloroform based DNA extraction, magnetic bead isolation, and silica gel column isolation.

In some embodiments, the identifying and characterizing methylation and/or hydroxymethylation comprises use of a methylation-specific PCR method.

In some embodiments, the identifying and characterizing methylation and/or hydroxymethylation comprises detection with a MassARRAY (Agena) method.

In some embodiments, the identifying and characterizing methylation and/or hydroxymethylation comprises use of a microarray hybridization technology.

In some embodiments, the identifying and characterizing methylation and/or hydroxymethylation comprises use of a sequencing-based method to analyze the distribution of 5-methylcytosine or 5-hydroxymethylcytosine by whole genome bisulfite sequencing or targeted methylation sequencing, preferably in combination with bisulfite treatment.

In some embodiments, a method for inferring and/or determining the polynucleotide variations comprises use of bioinformatics analysis, wherein the bioinformatics analysis comprises determination of optimal biomarkers and/or models by Spearman analysis or Pearson analysis, and preferably modeling is performed by Random Forest, LASSO regression, Logistic Regression, or deep-learning network.

In some embodiments, the method further comprises a simple quantitative detection performed after identifying and characterizing the methylation and/or hydroxymethylation biomarkers, wherein the quantitative detection comprises a methylation specific primer extension-based method, methylation-specific PCR (MSP), methylation-specific qPCR analysis, MassARRAY, and targeted methylation sequencing, based on the candidate markers selected via a high-throughput method.

In some embodiments, genes with the SNVs comprise at least one of genes AKT1, ALK, APC, AR, ARF, ARID1A, ATM, BRAF, BRCA1, BRCA2, CCND1, CCND2, CCNE1, CDH1, CDK4, CDK6, CDKN2A, CTNNB1, DDR2, EGFR, ERBB2, ESR1, EZH2, FBXW7, FGFR1, FGFR2, FGFR3, GATA3, GNA11, GNAQ, GNAS, HNF1A, HRAS, IDH1, IDH2, JAK2, JAK3, KIT, KRAS, MEK1, MEK2, ERK2, ERK1, MET, MLH1, MPL, MTOR, MYC, NF1, NFE2LE, NOTCH1, NPM1, NRAS, NTRK1, NTRK3, PDGFRA, PI3CA, PTEN, PTPN11, RAF1, RB1, RET, RHEB, RHOA, RIT1, ROS1, SMAD4, SMO, STK11, TERT, TP53, TSC1, and VHL.

In some embodiments, the polynucleotide with the InDels comprises at least one of genes ATM, APC, ARID1A, BRCA1, BRCA2, CDH1, CDKN2A, EGFR, ERBB2, GATA3, KIT, MET, MLH1, MTOR, NF1, PDGFRA, PTEN, RB1, SMAD4, STK11, TP53, TSC1, and VHL.

In some embodiments, the polynucleotide with the fusions comprises at least one of genes ALK, FGFR2, FGFR3, NTRK1, RET, ROS1, and EML4.

In some embodiments, the polynucleotide with the CNVs comprises genes AR, BRAF, CCND1, CCND2, CCNE1, CDK4, CDK6, EGFR, ERBB2 (HER2), FGFR1, FGFR2, KIT, KRAS, MET, MYC, PDGFRA, PI3CA, and RAF1.

In some embodiments, the inferring and/or determining polynucleotide variations comprises detecting gene amplification (CNV) of ERBB2 (HER2).

In the present invention, polynucleotide variations are detected by methylation and hydroxymethylation surrogate markers. As a non-invasive adjuvant diagnostic method for precision cancer medicine, the method for detecting polynucleotide variations of the present invention can be used for the detection of samples from a variety of sources and is particularly effective for the identification of surrogate biomarkers in blood. According to the method of the present invention, the detection may be completed by extracting only 1 ng free DNAs from plasma or serum (equivalent to 0.5 ml blood sample). The detection of the polynucleotide variations in the present invention can be used in the detection, prediction, precise treatment or postoperative monitoring of diseases.

### BRIEF DESCRIPTION OF THE DRAWINGS

FIG. 1 is a flow diagram of a method for detecting polynucleotide variations in an embodiment of the present invention.

FIG. 2 is a flow diagram showing a non-invasive methylation-based process for detecting ERBB2 (HER2) amplification in gastric cancer.

FIG. 3 shows identification of methylation biomarker-associated ERBB2 (HER2) amplification from tissue samples with gastric cancer.

FIG. 4 shows a simplified process for detecting ERBB2 (HER2) amplification by methylation-specific qPCR.

FIG. 5 shows the effectiveness of detecting ERBB2 (HER2) amplification in independent tissue samples by methylation-specific qPCR analysis.

FIG. 6 shows the effectiveness of detecting ERBB2 (HER2) amplification in gastric and breast cancer cell strains by methylation-specific qPCR.

FIG. 7 shows the effectiveness of detecting ERBB2 (HER2) amplification in plasma with gastric cancer by methylation-specific qPCR.

FIG. 8 is the average AUC of results from test sets of Logistic Regression modeling analysis in Example 2.

FIG. 9 is the average AUC of results from test sets of Random Forest modeling analysis in Example 2.

FIG. 10 is the average AUC of results from test sets in Example 3.

FIG. 11 is the average AUC of results from test sets in Example 4.

FIG. 12 is the average AUC of results from test sets in Example 5.

### DETAILED DESCRIPTION OF THE INVENTION

The experimental methods not marked with specific conditions in the following embodiments of the present invention shall be generally subjected to conventional conditions, or conditions recommended by the manufacturer. Various common chemical reagents used in the embodiments are commercially available.

Unless otherwise defined, all the technical and scientific terms used herein have the same meanings as commonly understood by a person skilled in the art to which the present invention belongs. The terms used in the description of the present invention are merely for the purpose of describing detailed embodiments but not construed as limiting the present invention.

The terms “comprise” and “have” and any variations thereof herein are intended to cover a non-exclusive inclusion. For example, a process, a method, an apparatus, a product, or a device that comprises a series of steps is not limited to the listed steps or components, but optionally further comprises other steps not listed, or optionally further comprises other steps or components inherent to the process, method, product, or apparatus.

“A plurality of” mentioned herein means two or more. The wording “and/or” describes a correlation of associated objects and means that there are three relationships, e.g. A and/or B may represent three cases, i.e., A alone, A and B together, and B alone. The character “/” generally indicates that there exists a correlation of “or” between the contextual objects.

Unless otherwise specified or defined herein, the terms “first, second . . . ” are merely intended to distinguish one name from another and do not represent a specific quantity or order.

For the convenience of understanding the present invention, the present invention will be described more comprehensively below. The present invention may be embodied in many different forms and shall not be limited the embodiments described herein. On the contrary, these embodiments are provided such that this disclosure will be understood more thoroughly and completely.

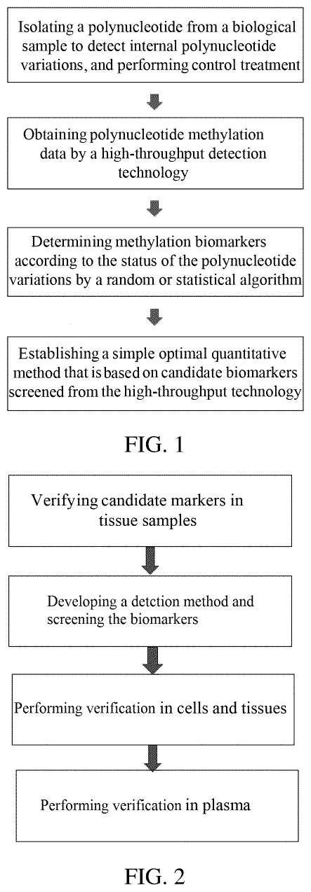

The present invention provides a method for detecting polynucleotide variations including SNVs, InDels, fusions, and CNVs in a biological sample. The method comprises sample preparation, or nucleic acid extraction and isolation from a biological sample; subsequent high-throughput methylation and/or hydroxymethylation analysis on polynucleotides by techniques known in the art; identification for optimally associated methylation and/or hydroxymethylation markers by means of bioinformatics tools; and/or building a model to infer the SNVs, InDels, fusions, and CNVs. The method may further comprise a database or collection of different methylation and/or hydroxymethylation features of various diseases as an additional reference to aid in the detection of methylation and/or hydroxymethylation biomarkers; and subsequent simplification and optimization of the detection techniques for quantification of methylation and/or hydroxymethylation surrogate biomarkers. Therefore, the present invention provides a method for detecting polynucleotide variations (FIG. 1), which may be used for early diagnosis, concomitant diagnosis and prognosis of genetic diseases.

As used herein, the term “polynucleotide” includes any related biopolymer. The polynucleotide includes, but is not limited to: DNA, RNA, amplicons, cDNA, dsDNA, ssDNA, plasmid DNA, cosmid DNA, high molecular weight (MW) DNA, chromosomal DNA, genomic DNA, viral DNA, bacterial DNA, mtDNA (mitochondrial DNA), mRNA, rRNA, tRNA, nRNA, siRNA, snRNA, snRNA, scaRNA, microRNA, dsRNA, ribozymes, riboswitches, and viral RNA (e.g. retroviral RNA).

As used herein, the term “biological sample” may be derived from a variety of sources, including human, mammals, non-human mammals, apes, monkeys, chimpanzees, reptiles, amphibians or birds, and the like. The biological sample may be in any form, such as 1) tissue-based, including but not limited to, fresh frozen tissues, formalin-fixed paraffin-embedded (FFPE) tissue specimens; 2) body fluid materials from animals, including but not limited to blood, serum, plasma, vitreous, sputum, urine, tear, sweat, saliva, semen, mucosal excrement, mucus, spinal fluid, amniotic fluid, and lymph. Those free polynucleotides may be derived from fetus (fluid extracted from a pregnant subject) or from the subject's own tissues; and 3) a cell line.

Isolation, purification, and preparation of a polynucleotide may be performed by various technologies known in the art. Suitable methods include those described herein in the embodiments, as well as variations thereof, including but not limited to treatment with a proteinase K, followed by phenol and/or chloroform extraction or commercial kits [Laird, P. W., et al., Simplified mammalian DNA isolation procedure. 1991. 19(15): p. 4293], and isolation methods based on isolation column or microparticles (or magnetic bead isolation) provided by Sigma-Aldrich, Life Technologies, Qiagen, Promega, Affymetrix, IBI and other similar companies. Kits and extraction methods may also be non-commercial. Generally, polynucleotides are first extracted by an isolation technology, for example, free DNA isolated from cells and other insoluble components of a biological sample. The isolation technology may include, but is not limited to, centrifugation or filtration or the like. After addition of buffers and other specific washing steps for different kits, DNA may be precipitated using an isopropanol precipitation method. A further washing step, for example, a silica gel column, may then be used to remove contaminants or salts. This general step may be optimized for a particular application. The objective of this step is to allow the purification of DNA or RNA in a larger amount of samples and to increase the amount of polynucleotide materials (DNA or RNA in most cases) available for detection, thereby facilitating analysis and improving accuracy.

In some embodiments, after being isolated and before being analyzed by a downstream high-throughput analysis technology (for example, a sequencing-based method), the polynucleotide may be premixed with one or more additional materials or reagents (e.g., a ligase, protease, restriction enzyme, polymerase, and the like).

In some embodiments, nucleotides isolated from a sample may also be amplified. For example, standard nucleic acid amplification systems are used, including PCR, ligase chain reaction, nucleic acid sequence-based amplification (NASBA), isothermal amplification (e.g., multiple displacement amplification (MDA), and helicase-dependent amplification (HDA)), branched DNA methods, and the like. Preferred amplification methods generally include PCR.

After being extracted and isolated from a biological sample, the polynucleotide is subjected to a treatment to determine whether the polynucleotide is methylated at a given site. This treatment may be in any form, including chemical or enzymatic conversion methods. Preferred chemical conversion method includes commercial or non-commercial bisulfite treatments. The enzymatic conversion method may be commercial or non-commercial TET-APOBEC-based conversions. After the conversion, methylation analysis may be performed to determine the methylation status of multiple CpG sites in the polynucleotide sequence. To achieve the purpose, various biotechnologies known in the art may be employed, including but not limited to: 1) microarray hybridization technologies, e.g. Illumina's Infinium HumanMethylation450 BeadChip (HM450K), Infinium CytoSNP-850K BeadChip or any custom-made array (Affymetrix) etc. [Sandoval, J., et al., Validation of a DNA methylation microarray for 450,000 CpG sites in the human genome. 2011. 6(6): p. 692-702.]; and 2) analysis of 5-methylcytosine distribution by a sequencing-based method in combination with bisulfite treatment. Sequencing methods may include, but are not limited to: Sanger sequencing, high-throughput sequencing, pyrosequencing, synthetic sequencing, single-molecule sequencing, nanopore sequencing, semiconductor sequencing, sequencing by ligation, sequencing by hybridization, digital gene expression (Helicos), next generation sequencing, single molecule synthetic sequencing (SMSS) (Helicos), massively parallel sequencing, clonal single molecule arrays (Solexa/Illumina), shotgun sequencing, Maxim Gilbert sequencing, primer walking, sequencing using PacBio, SOLiD, ion Torrent or nanopore platforms and any other sequencing methods known in the art. In some cases, sequencing methods may include multiple sample treatment units. The sample treatment units may include, but are not limited to, multi-channel devices, multi-path devices, multi-well devices, or other devices capable of processing multiple sample sets simultaneously. In addition, the sample treatment unit may include a plurality of sample chambers to allow multiple samples to be treated simultaneously. In some embodiments, a plurality of different types of free polynucleotides may be sequenced. The nucleic acid may be a polynucleotide or an oligonucleotide, including but not limited to DNA or RNA.

The subsequent analysis of polynucleotides using bioinformatics tools is related to two parts: 1) converting raw data from the high-throughput platform to a relative quantitative assay, which will allow downstream calculations and analysis for changes. These relevant bioinformatics tools have been established in the art. For example, array-based data, e.g., HM450K data from Illumina, typically quantifies the relative abundance of methylated and unmethylated sites by fluorescence intensity, and may be converted using software provided by Illumina; and data of bisulfite conversion, for example, from the whole genome bisulfite sequencing or targeted methylation bisulfite sequencing, involves methylation calling for individual Cs and requires statistical testing to assess differential methylation: including sequencing adapter adjustment, quality assessment on sequencing reads, reference genome-based calibration, and calculation and assessment of methylation degree. Lots of tools have been developed on the market, including but not limited to Cutadapt (trimming) [Martin, M. J. E. j., Cutadapt removes adapter sequences from high-throughput sequencing reads. 2011. 17(1): p. 10-12.], Bismark (calibration) [Krueger, F. and S. R. J. b. Andrews, Bismark: a flexible aligner and methylation caller for Bisulfite-Seq applications. 2011. 27(11): p. 1571-1572.], UCSC genome browser (data visualization), and methygo (post-alignment analysis). Quantitative measurements like beta-value (β) typically assess the methylation level by the ratio of intensities between methylated and unmethylated alleles; 2) determining optimal methylation markers for characterizing DNA mutations and other variations, which may be achieved by a simple correlation analysis (e.g., Spearman analysis or Pearson analysis) of a single biomarker, or by modeling using multiple biomarkers simultaneously with, for example, Random Forest Regression [Liaw, A. and M. J. R. n. Wiener, Classification and regression by randomForest. 2002. 2(3): p. 18-22.], LASSO regression [Tibshirani, R. J. J. o. t. R. S. S. S. B., Regression shrinkage and selection via the lasso. 1996. 58(1): p. 267-288.], Logistic Regression and deep learning neural networks.

In some embodiments, after marker identification, the detection of targeted methylation and/or hydroxymethylation pattern may be optimized to a simple quantitative method using existing technologies, including but not limited to oligonucleotide arrays, massARRAY, MS-based primer extension methods, methylation-specific PCR (MSP), and methylation-specific qPCR analysis. Among them, MSP is a mature technology for detecting the degree of gene methylation in selected gene sequences [Herman, J. G., et al., Methylation-specific PCR: a novel PCR assay for methylation status of CpG islands. 1996. 93(18): p. 9821-9826.]. Methylation-specific qPCR detection is a high-throughput quantitative methylation detection method, in which methylated and unmethylated DNAs are distinguished by a real-time fluorescent PCR technology using PCR primers (TaqMan.®), and the method requires no additional operations such as electrophoresis and hybridization at the end of PCR amplification, thereby reducing contamination and operating errors [Eads, C. A. et al., MethyLight: a high-throughput assay to measure DNA methylation. 2000. 28(8): p. e32-00.]. This real-time quantitative PCR comprises a methylation-sensitive probe complementary to the methylation site to be detected, for example, using a TaqMan probe. With the differential methylation status of the target sequence, only the TaqMan probe which is fluorescently labeled and is specific to bisulfite-converted methylated DNA may hybridize with a substrate nucleotide to release a fluorescent signal. The signal intensity is in direct proportion to the amount of PCR products, and accordingly the methylation degree of the sample can be calculated.

The present invention will be described comprehensively below in conjunction with examples, but is not limited to these examples.

EXAMPLE 1 ANALYSIS ON AMPLIFICATION STATUS OF ERBB2 (HER2) IN PLASMA AND TISSUE SAMPLES WITH GASTRIC CANCER BY METHYLATION BIOMARKERS

Gastric cancer is the fifth most common cancer in the word, and the second in Asia. Human epithelial factor receptor 2 (ERBB2 (HER2)) gene is amplified or overexpressed in 9% to 38% of gastric cancer patients [Rüschoff, J., et al., HER2 testing in gastric cancer: a practical approach. 2012. 25(5): p. 637-650.]. A phase III study of trastuzumab for treatment of gastric cancer (ToGA) shows that the combined treatment of chemotherapy with Trastuzumab (a monoclonal HER2 inhibiting antibody) improves survival rate relative to chemotherapy alone [Van Cutsem, E., et al., Efficacy results from the ToGA trial: A phase III study of trastuzumab added to standard chemotherapy (CT) in first-line human epidermal growth factor receptor 2 (HER2)-positive advanced gastric cancer (GC). 2009. 27(18_suppl): p. LBA4509-LBA4509.]. Trastuzumab is used as a standard targeted therapeutic drug for HER2-positive gastric cancer, which improves the importance of HER2 detection.

According to the National Comprehensive Cancer Network Oncology Clinical Practice Guidelines (NCCN Guidelines), tumor tissues should be subjected to assessment on HER2 overexpression and/or amplification by immunohistochemistry (IHC) and fluorescence or silver in situ hybridization (FISH or SISH), [Carlson, R. W., et al., HER2 testing in breast cancer: NCCN Task Force report and recommendations. 2006. 4(S3): p. S-1-S-22]. The IHC method is more popular for the detection of HER2 protein expression due to its cost and operation recommendation, while the FISH/SISH method is a gold standard for detecting the CNV status of HER2 gene. Researches show that as for the detection of HER2, the clinical IHC is highly correlated to FISH, and is a well-accepted method to detect the expression variation of HER2 [Vincent-Salomon A, MacGrogan G, Couturier J, et al: Calibration of immunohistochemistry for assessment of HER2 in breast cancer: results of the French Multicentre GEFPICS* Study. 42:337-347, 2003] [1. Furrer D, Jacob S, Caron C, et al: Concordance of HER2 immunohistochemistry and fluorescence in situ hybridization using tissue microarray in breast cancer. 37:3323-3329, 2017] [Arnould, L., et al., Accuracy of HER2 status determination on breast core-needle biopsies (immunohistochemistry, FISH, CISH and SISH vs FISH. 2012. 25(5): p. 675-682].

However, since most patients with gastric cancer are diagnosed as unresectable, advanced or metastatic cancer, it is difficult to obtain sufficient tissues for HER2 detection [Hofmann, M., et al., Assessment of a HER2 scoring system for gastric cancer: results from a validation study. 2008. 52(7): p. 797-805.]. At the same time, since pathological tissues of gastric cancer have higher heterogeneity, the conventional methods such as tissue biopsy, immunohistochemical staining and in situ hybridization detection require a higher skill level for sample collection, sample size and treatment. Moreover, multiple sampling may cause certain damage to the patients. Some other problems are constantly emerging in practical detection, for example, HER2 detection from gastroscopic biopsy has not been popularized, and the rate of detecting with in situ hybridization is low. As a result, the HER2 status in most cases of gastric cancer with immunohistochemical staining (IHC) 2+ was not determined. Further, HER2 positive rates from certain institutions are significantly different from those reported in domestic and foreign literatures. [Lee, H. E., et al., Clinical significance of intratumoral HER2 heterogeneity in gastric cancer. 2013. 49(6): p. 1448-1457.].

The present invention provides a non-invasive methylation-based method for analysis of HER2 amplification by liquid biopsy (FIG. 2).

### Patients

FFPE and plasma specimens of patients with gastric cancer were from the Department of Pathology of Southern Medical University. The project was approved by the Medical Ethics Committee of Southern Medical University. Informed consent was obtained from each patient. 2-5 FFPE glass slide samples were collected from each patient after surgery and 3-5 ml plasma was collected from each patient using a vacuum blood collection tube (BD, Cat #367525) prior to the surgery. The HER2 amplification status of each patient was confirmed by immunohistochemical staining, and was from official pathological reports of the hospital.

### Sample Collection and DNA Extraction

Genomic DNA was isolated from the FFPE tissue samples using a QIAamp-DNA-FFPE tissue kit (Qiagen, Cat #56404). Cell free DNA (cfDNA) was isolated from plasma using a Qiagen-Qiamp circulating nucleic acid kit (Qiagen, Cat #55114). Plasma was be kept away from repeated freezing and thawing to prevent the degradation of cfDNA. The concentration and mass of the cfDNA were determined by a Bioanalyzer 2100 (Agilent) via a Qubitds™ DNA HS assay kit (Thermo Fisher Scientific, Cat #Q32854) and an Agilent high-sensitivity DNA kit (Cat #5067-4626). Sequencing library was constructed for cfDNA with a yield greater than 3 ng and without excessive genomic DNA contamination.

### Bisulfite Conversion and Library Construction for Tissue Samples

The cfDNA bisulfite conversion was performed using a Zymo Lightning conversion reagent (Zymo Research, Cat #D5031) according to the instructions of the kit; after passing through a Zymo-Spin™ IC column, washing and desulfuration, the bisulfite-converted DNA was eluted twice with an M-elution buffer to a final volume of 17 μL.

For tissue samples, 2 ug genomic DNA was fragmented to about 200 bp (peak size) with a M220 focused-ultrasonicator (Covaris, Inc.) according to the instructions, afterwards, 800 ng of the purified fragmented genomic DNA was used for bisulfite conversion. After bisulfite conversion and purification, the bisulfite-converted DNA was quantified by NanoDrop (Thermo Fisher Scientific) at A260. 150 ng of the bisulfite-converted products were then used for the library preparation of FFPE tissue samples.

NGS pre-library preparation was completed by an AnchorDx-Epivision™ methylation library preparation kit (AnchorDx, Cat #A0UX00019) and an AnchorDx-EpiVisio™ index PCR kit (AnchorDx, Cat #A2DX00025). The amplified DNA was purified using 1:6 Agencourt AMPure XP magnetic beads (Beckman Coulter, Cat #A63882) after end pair reparation, 3′-terminal adapter ligation, and reverse complementary DNA amplification. The amplified pre-library was purified with XP magnetic beads after the 3′-terminal adapter of the reverse complementary DNA was ligated with the index PCR primers (i5 and i7). The DNAs containing more than 800 ng of the pre-hybridized library may be used for the subsequent targeted enrichment analysis.

Target enrichment was performed using an AnchorDx-Epivision™ target enrichment kit (AnchorDx, Cat #A0UX00031), a methylation panel and an AnchorDx BrGcMet panel. 1000 ng DNA containing up to 4 pre-hybridized libraries were pooled for targeted enrichment with an AnchorDx-BrGcMet methylation panel. The AnchorDx-BrGcMet panel included 12,892 pre-selected regions enriched for cancer-specific methylation, and the total size of the directed genomic region included 123,269 CpG sites. The procedures of the probe hybridization, purification and final PCR amplification followed a reported solution [Liang, W., et al., Non-invasive diagnosis of early-stage lung cancer using high-throughput targeted DNA methylation sequencing of circulating tumor DNA (ctDNA). 2019. 9(7): p. 2056.].

### DNA Sequencing and Calculation of DNA Methylation Level

The enrichment library was sequenced by the Illumina HiSeq X-Ten sequencing system according to the instructions. β value (β) was defined by a ratio of intensities between methylated and unmethylated alleles and used to estimate the methylation level. The β value is between 0 and 1 with 0 being unmethylated and 1 fully methylated [Du, P., et al., Comparison of Beta-value and M-value methods for quantifying methylation levels by microarray analysis. 2010. 11(1): p. 587.].

Establishment and Verification of the Methylation-Specific qPCR Detection Method in Plasma Samples

Methylation markers were designed and optimized for methylation-specific qPCR analysis (AnchorDx, China) according to the instructions. EpiTect PCR Control DNA Set (Qiagen, Germany) was set as a positive control and a negative control. qPCR reaction was performed on a QuantStudio 3 real-time PCR system (Thermo Fisher, USA) using an Epimark qPCR reaction system (NEB, Cat#M0490) under the following cycle conditions: denaturation at 98° C. for 30 s, 40 cycles (95° C. for 10 s, 62° C. for 20 s).

The recommended amount of the bisulfite-converted cfDNA for plasma samples was 10 ng. All the purified cfDNA were used for bisulfite conversion when the cfDNA yield was within 1-10 ng. After the bisulfite conversion, all the bisulfite-converted cfDNA were used for subsequent methylation-specific qPCR detection.

With regard to the methylation-specific qPCR analysis, ΔCt represents a co-methylation level in a target region, where ΔCt=mean Ct (target region)−mean Ct (internal control region). For the region without a definite Ct value, an artificial ΔCt will be designated as 35.

### Data Processing

A R-pROC software package was used for clinical performance analysis on individual markers and the final classification model. A logistic regression model was built using a PythonSklearn package. A student's t-test was used to statistically analyze the probability distribution of HER2 amplification in different test groups.

### Results

### Identification of HER2-Associated Methylation Surrogate Biomarkers in Amplified Tissue Materials

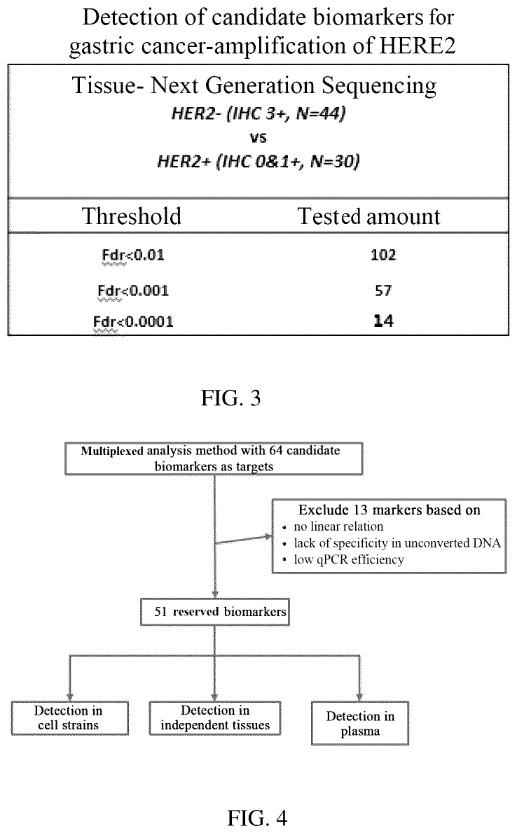

To identify specific methylation features of HER2 amplification status in gastric cancer, 74 FFPE tissue samples were collected, including 44 HER2− samples (IHC0 or 1+) and 33 HER2+ samples (IHC3+); all the samples were in the advanced stage (stage III or IV). A high-throughput targeted methylation sequencing method was used, and relevant cleanup, processing and analysis on the raw sequencing data were performed [Liang, W., et al., Non-invasive diagnosis of early-stage lung cancer using high-throughput targeted DNA methylation sequencing of circulating tumor DNA (ctDNA). 2019. 9(7): p. 2056.]. The percentage (β value) of methylated cytosine at each site was determined on the basis of the reads. Each site of the HER2+ sample and the HER2− sample was analyzed for statistical difference. 102 candidate methylation marker sites were identified. These markers were significantly different between the HER2− and HER2+ groups (FDR <0.01, FIG. 3).

Establishment of the Multiple Methylation-Specific qPCR Detection Method and Screening of Biomarkers

Subsequently, methylation-specific qPCR detection was designed, in which qPCR primers and probes were designed for 64 candidate markers within the 102 candidate markers (the difference in β value greater than 5% between the HER2+ and HER2− tissue samples, and accorded with the basic design principles of a primer [Davidović, R. S., et al., Methylation-specific PCR: four steps in primer design. 2014. 9(12): p. 1127-1139]). Diluted internal reference genes and unconverted DNA served as controls to detect the linearity and specificity of these detection methods, through which 13 biomarkers were excluded due to poor detection performance (FIG. 4).

### Verification in Tissue Samples, Cell Lines and Plasma Samples

The inventors further verified the optimized methylation-specific qPCR detection in 1) independent tissue samples, 2) gastric and breast cancer cell lines, and 3) gastric cancer plasma samples, so as to facilitate discovery of these markers.

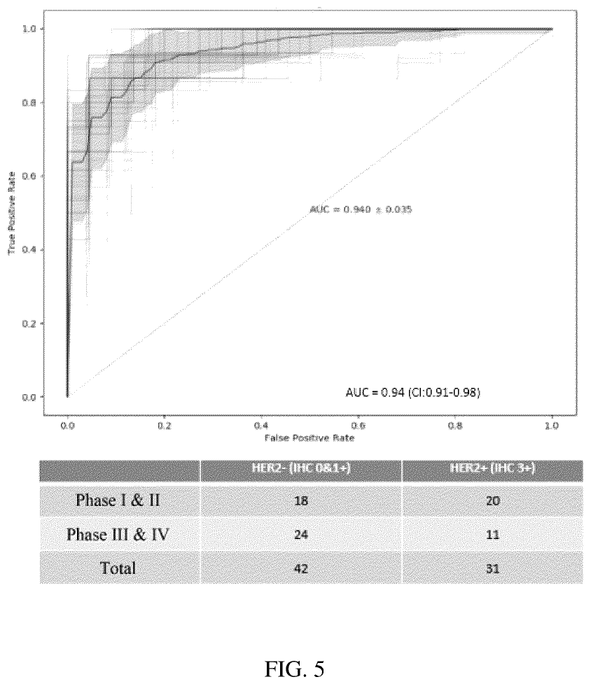

In independent FFPE-gastric cancer samples (42-HER2− vs 31-HER2+), a linear regression model based on 10 methylation markers was constructed by analysis modeling on the 64 candidate markers with a LASSO model package (R package-glmnet package); and the AUC of HER2 amplification was determined to be 0.94 (FIG. 5).

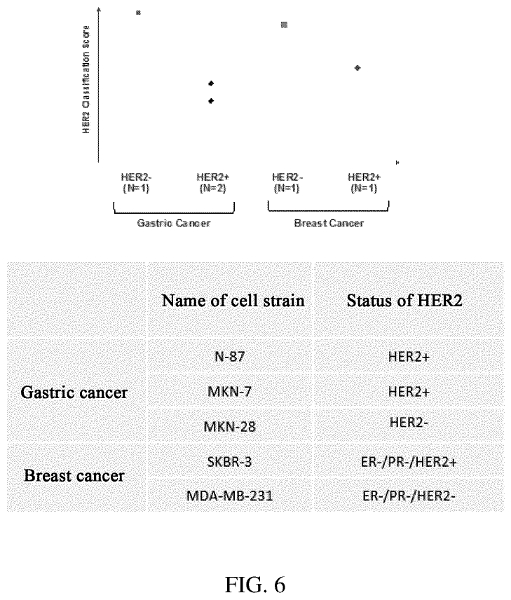

In the cell line samples, HER2 was scored according to the ΔCt values of the methylation biomarkers (the scoring was based on a linear regression model of 2 methylation markers). Based on the score, it was found that there was a significant difference between gastric cancer HER2+ and HER2− cell lines as well as breast cancer HER2+ and HER2− cell lines (FIG. 6).

In plasma samples, three different modeling methods were tested (least absolute shrinkage and selection operator (LASSO) [Tibshirani, R. J. J. o. t. R. S. S. S. B., Regression shrinkage and selection via the lasso. 1996. 58(1): p. 267-288.], Random Forest (RF) [Liaw, A. and M. J. R. n. Wiener, Classification and regression by randomForest. 2002. 2(3): p. 18-22.] and Linear Regression (LR) [Long, J. S. and L. H. J. T. A. S. Ervin, Using heteroscedasticity consistent standard errors in the linear regression model. 2000. 54(3): p. 217-224.]) models.

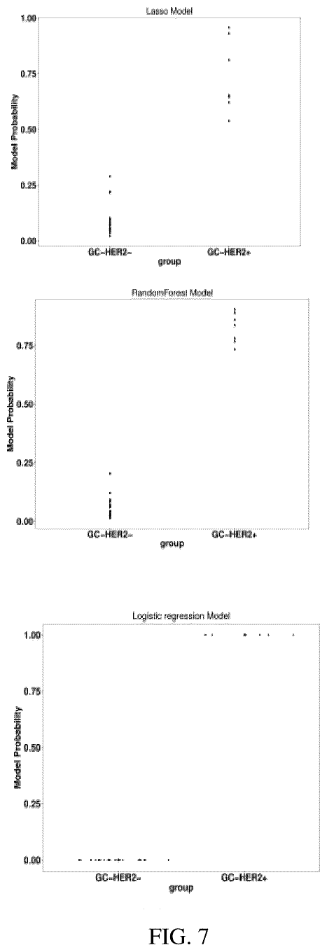

As expected, there was also a significant difference between the gastric cancer HER2− plasma samples (N=7) and the HER2− plasma samples (N=20) based on the HER2 classification score (FIG. 7).

TABLE 1
Package information used for modeling

| | | | Name of R |
|---|---|---|---|
| | | Important | language |
| | Model feature | index setting | package |
| LASSO | the high colinearity | family = “binomial”, | glmnet |
| | influence among | alpha = 1, | package |
| | the characteristic | lambda = | |
| | variables in | lambda.1se, | |
| | traditional linear | nfold = 10 | |
| | models is eliminated | | |
| | mainly by adding | | |
| | an offset item, thus | | |
| | the variance of the | | |
| | model is reducing | | |
| RF | multiple decision-making | importance = | randomForest |
| | tree models | TRUE, | |
| | are established and | ntree = 100, | |
| | merged to obtain a | mtry = 2 | |
| | more accurate and | | |
| | stable model, which, | | |
| | compared to linear | | |
| | models, is easier to | | |
| | find the complex | | |
| | correlation between | | |
| | characteristic variables, | | |
| | but may overfit | | |
| | on some sample | | |
| | sets having higher | | |
| | noise | | |
| LR | A linear model; | Family = | glmnet |
| | output is a linear | binomial | package |
| | combination of input | (link = ‘logit’) | |
| | variables, and the | | |
| | model is very | | |
| | sensitive to abnormal | | |
| | values | | |

To sum up, these data indicate that free methylation biomarkers may be used to accurately assess the HER2 amplification status of a patient with gastric cancer and thus may serve as a concomitant diagnostic product for the targeted therapy of gastric cancer.

### EXAMPLE 2 ANALYSIS ON INDELs OF THE GENE ERBB2 IN TISSUE SAMPLES WITH LUNG CANCER BY METHYLATION BIOMARKERS

Lung cancer is a cancer with the highest incidence rate and mortality in the world, and is the largest cancer in China. ERBB2 (HER2) gene belongs to a family of human epidermal growth factor receptors (HER). HER2 gene mutations are widely present in many solid tumors, including breast cancer, gastric cancer, lung cancer, and the like. ERBB2 mutation is one of the common driving mutation genes in lung cancer, and can be detected in 2-4% of lung cancers, and exon 20/INDEL is the most common mutation, which can activate kinase activity and downstream signaling pathways, and promote cell survival and tumorigenesis [Wang S E, et al. HER2 kinase domain mutation results in constitutive phosphorylation and activation of HER2 and EGFR and resistance to EGFR tyrosine kinase inhibitors. 2006; 10(1):25-38.].

### Patients

FFPE samples of patients with lung cancer were from the First Affiliated Hospital of Guangzhou Medical University. The project was approved by the Medical Ethics Committee of the First Affiliated Hospital of Guangzhou Medical University. Informed consent was obtained from each patient. 2-5 FFPE glass slide samples were collected from each patient after surgery, and the patient's relevant personal pathological information was obtained from the official pathological report of the hospital.

### Whole Genome Sequencing Analysis of Tissue Samples

FFPE tissue samples were subjected to whole genome sequencing analysis by a third party (Mingma Technologies).

As for DNA extraction, bisulfite conversion, library construction and methylation sequencing of the tissue samples, please refer to Example 1 for detail.

### Results

### Identification of ERBB2 EXON 20 INDEL-Associated Methylation Surrogate Biomarkers in Lung Cancer Tissues and Modeling Analysis

To identify specific methylation features of the ERBB2 INDEL status in gastric cancer, 78 collected FFPE tissue samples were subjected to whole genome INDEL analysis. It was found that 18 samples had INDEL mutations at ERBB2 EXON20, and the remaining 60 samples were normal (see Table 2).

TABLE 2

| | chr7 | pos | | gene | type | band |
|---|---|---|---|---|---|---|
| B2-C-028 | chr7 | 55242464 | exonic | EGFR | nonframeshift | 7p11.2 |
| | | | | | deletion | |
| B5-C-036 | chr7 | 55242464 | exonic | EGFR | nonframeshift | 7p11.2 |
| | | | | | deletion | |
| B3-C-018 | chr7 | 55242464 | exonic | EGFR | nonframeshift | 7p11.2 |
| | | | | | deletion | |
| B2-C-007 | chr7 | 55242464 | exonic | EGFR | nonframeshift | 7p11.2 |
| | | | | | deletion | |
| B3-C-039 | chr7 | 55242464 | exonic | EGFR | nonframeshift | 7p11.2 |
| | | | | | deletion | |
| B2-C-027 | chr7 | 55242464 | exonic | EGFR | nonframeshift | 7p11.2 |
| | | | | | deletion | |
| B2-C-023 | chr7 | 55242464 | exonic | EGFR | nonframeshift | 7p11.2 |
| | | | | | deletion | |
| B3-C-026 | chr7 | 55242464 | exonic | EGFR | nonframeshift | 7p11.2 |
| | | | | | deletion | |
| B2-C-029 | chr7 | 55242465 | exonic | EGFR | nonframeshift | 7p11.2 |
| | | | | | deletion | |
| B4-C-006 | chr7 | 55242466 | exonic | EGFR | nonframeshift | 7p11.2 |
| | | | | | deletion | |
| B3-C-081 | chr7 | 55242466 | exonic | EGFR | nonframeshift | 7p11.2 |
| | | | | | deletion | |
| B2-C-018 | chr7 | 55242469 | exonic | EGFR | nonframeshift | 7p11.2 |
| | | | | | deletion | |
| B4-C-021 | chr7 | 55242469 | exonic | EGFR | nonframeshift | 7p11.2 |
| | | | | | deletion | |
| B5-C-038 | chr7 | 55242469 | exonic | EGFR | nonframeshift | 7p11.2 |
| | | | | | deletion | |
| B3-C-067 | chr7 | 55242469 | exonic | EGFR | nonframeshift | 7p11.2 |
| | | | | | deletion | |
| B3-C-068 | chr7 | 55248998 | exonic | EGFR | nonframeshift | 7p11.2 |
| | | | | | insertion | |
| B3-C-040 | chr7 | 55249002 | exonic | EGFR | nonframeshift | 7p11.2 |
| | | | | | insertion | |
| B3-C-048 | chr7 | 55249010 | exonic | EGFR | nonframeshift | 7p11.2 |
| | | | | | insertion | |

A high-throughput targeted methylation sequencing method was used, and relevant cleanup, processing and analysis on the raw sequencing data were performed [Liang, W., et al., Non-invasive diagnosis of early-stage lung cancer using high-throughput targeted DNA methylation sequencing of circulating tumor DNA (ctDNA). 2019. 9(7): p. 2056.]. The percentage (β value) of methylated cytosine at each site was determined on the basis of the reads. Each site of the ERBB2 EXON20 INDEL+ sample and ERBB2 EXON20 INDEL− sample was analyzed for statistical difference. The condition of p value <0.001. fdr<0.1, and |diff|>0.1 was used to identify 5 candidate methylation marker sites with significant difference.

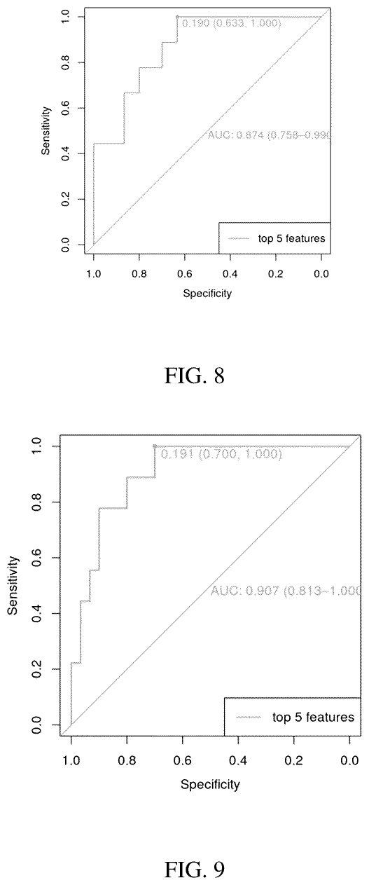

78 samples were grouped by 7:3 and divided by 100 times; the 5 candidate methylation markers were subjected to logistic regression modeling analysis; and the average AUC of results from test sets may be up to 0.874 (FIG. 8).

78 samples were grouped by 7:3 and divided by 100 times; the 5 candidate methylation markers were subjected to Random Forest modeling analysis; and the average AUC of results from test sets may be up to 0.907 (FIG. 9). The results indicate that the models using the 5 markers may accurately distinguish the status of the EXON 20 INDEL mutation status of the ERBB2 gene in the samples.

### EXAMPLE 3 ANALYSIS ON ATM FUSIONS IN TISSUE SAMPLES WITH LUNG CANCER BY METHYLATION BIOMARKERS

Lung cancer is a cancer with the highest incidence rate and mortality in the world, and is the largest cancer in China. The protein encoded by the ATM gene belongs to a PI3/PI4 kinase family, and it is an important cell cycle checkpoint kinase that regulates a series of downstream proteins through phosphorylation, including cancer suppressor proteins p53 and BRCA1, checkpoint kinase CHK2, checkpoint proteins RAD17 and RADS as well as DNA repair protein NBS1. The protein generally involves in DNA damage repair process and maintenance of genome stability.

Mutations in the ATM gene are closely related to the occurrence of lung cancer. Researches show that there is a strong correlation between the ATM gene and the sensitivity of a tumor to radiotherapy. At the same time, the mutation status of the ATM kinase in lung cancer cells may be used as a novel tumor marker to measure the sensitivity of a patient to drugs like MEK inhibitors, which may greatly improve the diagnosis and subsequent treatment effect for patients of this subtype, and may expand the use of such drugs in tumor patients with a mutation in genes other than RAS and BRAF [Ji X, et al. Protein-altering germline mutations implicate novel genes related to lung cancer development. 2020; 11(1):1-14.].

### Patient

FFPE samples of patients with lung cancer were from the First Affiliated Hospital of Guangzhou Medical University. The project was approved by the Medical Ethics Committee of the First Affiliated Hospital of Guangzhou Medical University. Informed consent was obtained from each patient. 2-5 FFPE glass slide samples were collected from each patient after surgery, and the patient's relevant personal pathological information was obtained from the official pathological report of the hospital.

### Whole Genome Sequencing Analysis of Tissue Samples

FFPE tissue samples were subjected to whole genome sequencing analysis by a third party (Mingma Technologies).

As for DNA extraction, bisulfite conversion, library construction and methylation sequencing of the tissue samples, please refer to Example 1 for detail.

### Results

### Identification of ATM FUSION-Associated Methylation Surrogate Biomarkers in Lung Cancer Tissues and Modeling Analysis

To identify specific methylation features of the ATM FUSION status in lung cancer, 6 FFPE tissue samples with ATM fusions (ATM FUSION+) and 20 samples without ATM fusions (ATM FUSION−) were collected (verified by whole genome sequencing and analysis).

A high-throughput targeted methylation sequencing method was used, and relevant cleanup, processing and analysis on the raw sequencing data were performed [Liang, W., et al., Non-invasive diagnosis of early-stage lung cancer using high-throughput targeted DNA methylation sequencing of circulating tumor DNA (ctDNA). 2019. 9(7): p. 2056.]. The percentage (β value) of methylated cytosine at each site was determined on the basis of the reads. Each site of the ATM FUSION+ sample and ATM FUSION− sample was analyzed for statistical difference. The condition of p value <0.001 and fdr<0.05 was used to identify 4 candidate methylation marker sites with significant difference.

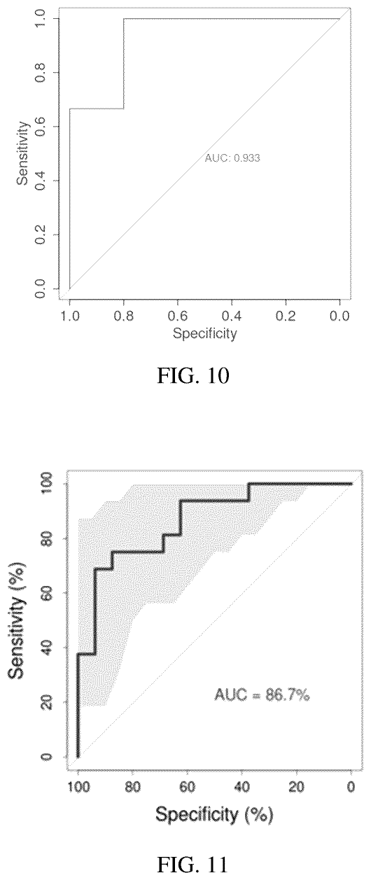

26 samples were grouped by 5:5 and divided by 50 times; the 4 candidate methylation markers were subjected to Random Forest modeling analysis; the average AUC of results from test sets may be up to 0.933 (FIG. 10). The results indicate that the model using the 4 markers may accurately distinguish the fusion status of the ATM gene in the samples.

### EXAMPLE 4 ANALYSIS ON THE EGFR EXON 21 L858R POINT MUTATION (SNV) IN TISSUE SAMPLES WITH BY METHYLATION BIOMARKERS

Lung cancer is a cancer with the highest incidence rate and mortality in the world, and is the largest cancer in China.

EGFR is a member of the HER family and is widely distributed on the surface of mammalian epithelial cells, fibroblasts, glial cells and keratinocytes. The EGFR signaling pathway plays an important role in the growth, proliferation, differentiation and other physiological processes of cells.

EGFR is one of the most common driving genes in non-small cell lung cancer (NSCLC). In clinical practice, the detection of EGFR gene is typically used for the pre-treatment evaluation of patients with advanced NSCLC. The existence of EGFR mutation means that patients may have corresponding targeted drugs with an effective rate of 60%-70% and small side effects. The emergence of EGFR-TKI targeted drugs significantly improves the survival of patients with EGFR mutation-positive advanced lung cancer and enables the clinical treatment of lung cancer to enter the era of precise treatment.

The most common mutation site of the EGFR gene is located at exons 18-21, of which the exon 18 has a mutation of G719X; the exon 19 has a mutation of E19del; the exon 20 has mutations of T790M, S768I and E20ins, and the exon 21 has mutations of L858R and L861Q. Among them, the deletion mutation E19del of the exon 19 and the point mutation L858R of the exon 21 are the most common, and patients with these mutations are the major population for oral EGFR targeted drug therapy [Yamamoto H, Toyooka S, and Mitsudomi T J L c. Impact of EGFR mutation analysis in non-small cell lung cancer. 2009; 63(3):315-21.].

### Patients

FFPE samples of patients with NSCLC were from the First Affiliated Hospital of Guangzhou Medical University. The project was approved by the Medical Ethics Committee of the First Affiliated Hospital of Guangzhou Medical University. Informed consent was obtained from each patient. 2-5 FFPE glass slide samples were collected from each patient after surgery, and the patient's relevant personal pathological information was obtained from the official pathological report of the hospital.

### Whole Genome Sequencing Analysis of Tissue Samples

FFPE tissue samples were subjected to whole genome sequencing analysis by a third party (Mingma Technologies).

As for DNA extraction, bisulfite conversion, library construction and methylation sequencing of the tissue samples, please refer to Example 1 for detail.

### Result, Identification of EGFR EXON 21 L858R Point Mutation-Associated Methylation Surrogate Biomarkers in NSCLC Tissues and Modeling Analysis

To identify specific methylation features of the EGFR L858R point mutation status in lung cancer, 39 FFPE tissue samples with EGFR L858R point mutations (L858R+) and 39 samples without the point mutation (L858R−) were collected (verified by whole genome sequencing and analysis).

A high-throughput targeted methylation sequencing method was used, and relevant cleanup, processing and analysis on the raw sequencing data were performed [Liang, W., et al., Non-invasive diagnosis of early-stage lung cancer using high-throughput targeted DNA methylation sequencing of circulating tumor DNA (ctDNA). 2019. 9(7): p. 2056.]. The percentage (β value) of methylated cytosine at each site was determined on the basis of the reads. Each site of the L858R+ sample and L858R− sample was analyzed for statistical difference. The condition of p value <0.001, and fdr<0.05 was used to identify 20 candidate methylation marker sites with significant difference.

78 samples were grouped by 5:5 and divided by 50 times; the 20 candidate methylation markers were subjected to Random Forest modeling analysis; the average AUC of results from test sets may be up to 0.867 (FIG. 11). The results indicate that the model using the 20 markers may accurately distinguish the status of the EGFR L858R point mutation in the samples.

### EXAMPLE 5 Analysis ON POINT MUTATION (SNV) STATUS OF EXONS 5-8 of P53 GENE IN TISSUE SAMPLES WITH LUNG CANCER BY METHYLATION BIOMARKERS

Lung cancer is a cancer with the highest incidence rate and mortality in the world, and is the largest cancer in China.

P53 gene is an important cancer suppressor gene. Deletion or mutation has been found in the p53 gene in 50% of human tumors, and is closely related to the development and progression of tumors.

The p53 gene mutation is one of the important factors that lead to many tumors, including lung cancer. p53 gene mutation mainly includes point mutation and allelic deletion. It has been reported that in about 200 different tumors, 50% tumors carry a p53 gene mutation. 4 mutation hotspots located in exons 5-8 have been found in the p53 gene. Although the mutation profile of p53 gene is different in tumors occurring in different tissues and organs, about 90% of the mutations are located in this region. These mutation hotspots encode amino acids 132-143, 174-179, 236-248 and 272-281, respectively [Rodin S N, and Rodin A S J P o t N A o S . Human lung cancer and p53: the interplay between mutagenesis and selection. 2000; 97(22):12244-9.].

### Patients

FFPE samples of patients with lung cancer were from the First Affiliated Hospital of Guangzhou Medical University. The project was approved by the Medical Ethics Committee of the First Affiliated Hospital of Guangzhou Medical University. Informed consent was obtained from each patient. 2-5 FFPE glass slide samples were collected from each patient after surgery, and the patient's relevant personal pathological information was obtained from the official pathological report of the hospital.

### Whole Genome Sequencing Analysis of Tissue Samples

FFPE tissue samples were subjected to whole genome sequencing analysis by a third party (Mingma Technologies).

As for DNA extraction, bisulfite conversion, library construction and methylation sequencing of the tissue samples, please refer to Example 1 for detail.

### Results

### Identification of P53 EXON 5-8 Point Mutation Status-Associated Methylation Surrogate Biomarkers in Lung Cancer Tissues and Modeling Analysis

To identify specific methylation features of the P53 EXON 5-8 point mutation status in lung cancer, 40 FFPE tissue samples with P53 EXONS-8 point mutations and 38 samples without the point mutation were collected (verified by whole genome sequencing and analysis).

A high-throughput targeted methylation sequencing method was used, and relevant cleanup, processing and analysis on the raw sequencing data were performed [Liang, W., et al., Non-invasive diagnosis of early-stage lung cancer using high-throughput targeted DNA methylation sequencing of circulating tumor DNA (ctDNA). 2019. 9(7): p. 2056.]. The percentage (β value) of methylated cytosine at each site was determined on the basis of the reads. Each site of the P53 EXON 5-8 point mutation positive sample and negative sample was analyzed for statistical difference. The condition of p value <0.001 and fdr<0.05 were used to identify 20 candidate methylation marker sites with significant difference.

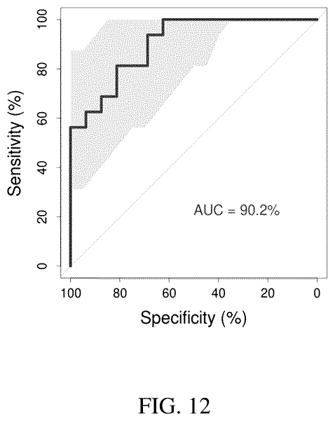

78 samples were grouped by 5:5 and divided by 50 times; the 20 candidate methylation markers were subjected to Random Forest modeling analysis; the average AUC of results from test sets may be up to 0.902 (FIG. 12). The results indicate that the model using the 20 markers may accurately distinguish the status of the EXON 5-8 point mutation of the P53 gene in the samples.

Although the preferred embodiments of the present invention have been specifically described above, the present invention is not limited to the embodiments. Those skilled in the art may further make equivalent modifications or substitutions without departing from the spirit of the present invention. These equivalent modifications or substitutions shall fall within the scope defined in the claims of the present application.

What is claimed is:

1. A method comprising:
(a) obtaining a polynucleotide from a biological sample;
(b) assaying the polynucleotide to detect methylation and/or hydroxymethylation biomarkers;
(c) using the detected methylation and/or hydroxymethylation biomarkers to train a machine learning model, wherein the machine learning model is configured to detect polynucleotide variations in the biological sample based at least in part on an analysis of methylation and/or hydroxymethylation biomarkers.

2. The method of claim 1, wherein the polynucleotide comprises deoxyribonucleic acid (DNA).

3. The method of claim 1, wherein the polynucleotide comprises ribonucleic acid (RNA).

4. The method of claim 1, wherein the polynucleotide variations comprise single-nucleotide variations (SNVs).

5. The method of claim 4, wherein the SNVs correspond to a gene selected from the group consisting of AKT1, ALK, APC, AR, ARF, ARID1A, ATM, BRAF, BRCA1, BRCA2, CCND1, CCND2, CCNE1, CDH1, CDK4, CDK6, CDKN2A, CTNNB1, DDR2, EGFR, ERBB2, ESR1, EZH2, FBXW7, FGFR1, FGFR2, FGFR3, GATA3, GNA11, GNAQ, GNAS, HNF1A, HRAS, IDH1, IDH2, JAK2, JAK3, KIT, KRAS, MEK1, MEK2, ERK2, ERK1, MET, MLH1, MPL, MTOR, MYC, NF1, NFE2LE, NOTCH1, NPM1, NRAS, NTRK1, NTRK3, PDGFRA, PI3CA, PTEN, PTPN11, RAF1, RB1, RET, RHEB, RHOA, RIT1, ROS1, SMAD4, SMO, STK11, TERT, TP53, TSC1, VHL, and a combination thereof.

6. The method of claim 1, wherein the polynucleotide variations comprise insertions and/or deletions (indels).

7. The method of claim 6, wherein the indels correspond to a gene selected from the group consisting of ATM, APC, ARID1A, BRCA1, BRCA2, CDH1, CDKN2A, EGFR, ERBB2, GATA3, KIT, MET, MLH1, MTOR, NF1, PDGFRA, PTEN, RB1, SMAD4, STK11, TP53, TSC1, VHL, and a combination thereof.

8. The method of claim 1, wherein the polynucleotide variations comprise fusions.

9. The method of claim 8, wherein the fusions correspond to a gene selected from the group consisting of ALK, FGFR2, FGFR3, NTRK1, RET, ROS1, EML4, and a combination thereof.

10. The method of claim 1, wherein the polynucleotide variations comprise copy number variations (CNVs).

11. The method of claim 10, wherein the CNVs correspond to a gene selected from the group consisting of AR, BRAF, CCND1, CCND2, CCNE1, CDK4, CDK6, EGFR, ERBB2 (HER2), FGFR1, FGFR2, KIT, KRAS, MET, MYC, PDGFRA, PI3CA, RAF1, and a combination thereof.

12. The method of claim 1, wherein the polynucleotide variations comprise an ERBB2 (HER2) gene amplification.

13. The method of claim 1, wherein the biological sample comprises a biological fluid sample.

14. The method of claim 13, wherein the biological fluid sample comprises blood, serum, plasma, vitreous body, sputum, urine, tear, sweat, or saliva.

15. The method of claim 1, wherein the biological sample comprises a tissue sample.

16. The method of claim 1, wherein the biological sample comprises a cell sample.

17. The method of claim 16, wherein the cell sample comprises a cell line sample.

18. The method of claim 1, wherein (a) comprises isolating the polynucleotide from the biological sample.

19. The method of claim 18, wherein the isolating comprises phenol-based and/or chloroform-based DNA extraction, magnetic bead isolation, or silica gel column isolation.

20. The method of claim 1, wherein (b) comprises performing a chemical conversion or enzymatic conversion.

21. The method of claim 20, wherein the chemical conversion comprises a bisulfite treatment.

22. The method of claim 20, wherein the enzymatic conversion method comprises use of ten-eleven translocation (TET)- apolipoprotein B mRNA editing enzyme (APOBEC) or TET enzyme plus pyridine borane.

23. The method of claim 1, wherein (b) comprises amplifying the polynucleotide.

24. The method of claim 23, wherein the amplifying comprises polymerase chain reaction (PCR).

25. The method of claim 24, wherein the PCR comprises methylation-specific PCR or a methylation-specific quantitative PCR (qPCR).

26. The method of claim 1, wherein (b) comprises use of mass spectrometry.

27. The method of claim 26, wherein the mass spectrometry comprises matrix-assisted laser desorption/ionization-time of flight (MALDI-TOF) mass spectrometry.

28. The method of claim 1, wherein (b) comprises use of microarray hybridization.

29. The method of claim 1, wherein (b) comprises sequencing the polynucleotide.

30. The method of claim 29, wherein the sequencing comprises whole genome bisulfite sequencing or targeted methylation sequencing.

31. The method of claim 30, wherein the whole genome bisulfite sequencing or the targeted methylation sequencing is performed in combination with bisulfite and/or enzymatic reagent treatment.

32. The method of claim 1, wherein (c) comprises selecting at least a subset of the detected methylation and/or hydroxymethylation biomarkers to derive an algorithm.

33. The method of claim 32, wherein the selecting comprises performing Statistical analysis, Spearman analysis or Pearson analysis.

34. The method of claim 1, wherein the algorithm derivation uses machine learning modeling.

35. The method of claim 34, wherein the machine learning model comprises a Random Forest, a LASSO regression, a Logistic Regression, or a deep-learning network.

36. The method of claim 1, wherein (b) comprises performing a methylation-specific primer extension-based assay.
